(19) United States
(12) Patent Application Publication
(10) Pub. No.: US 2026/0263330 A1
(43) Pub. Date: Sep. 10, 2026

(54) METHOD FOR PRODUCING A PRESERVED COSMETIC WATER-IN-OIL EMULSION FROM A GLYCERIN-IN-OIL EMULSION

(71) Applicant: BEIERSDORF AG, Hamburg (DE)

(72) Inventors: Josefine Jasmin KNORR, Hamburg (DE); Petra KOCH, Hamburg (DE); Kerstin SKUBSCH, Prisdorf (DE)

(73) Assignee: BEIERSDORF AG, Hamburg (DE)

(21) Appl. No.: 19/491,266

(22) PCT Filed: May 23, 2024
(86) PCT No.: PCT/EP2024/064228

§ 371 Date: Dec. 8, 2025

(30) Foreign Application Priority Data
Jun. 9, 2023 (DE) 10 2023 205 394.2

(51) Int. Cl. A61K 8/06; A61K 8/23; A61K 8/34; A61K 8/37; A61K 8/92

(52) U.S. Cl. CPC A61K 8/064; A61K 8/23; A61K 8/345; A61K 8/375; A61K 8/92; A61K 2800/524; A61K 2800/87; A61K 2800/882

(57) ABSTRACT

The invention relates to a method for producing a cosmetic water-in-oil emulsion, wherein a glycerin-in-oil emulsion containing a) glycerin, b) one or more lipids that are liquid at room temperature and under normal pressure, c) one or more waxes, d) one or more salts, e) polyglyceryl-3 polyricinoleate (INCI: polyglyceryl-3-polyricinoleate), and f) one or more preservatives, is mixed with water in a storage container and is shaken or stirred.

The present invention relates to a process for producing a preserved cosmetic water-in-oil emulsion from a cosmetic glycerin-in-oil emulsion comprising glycerin, one or more lipids that are liquid at room temperature and standard pressure, one or more waxes, salts and polyglyceryl-3 polyricinoleate (INCI: Polyglyceryl-3 Polyricinoleate), where the preparation comprises one or more preservatives.

The desire to look beautiful and attractive is naturally rooted in humans. Although ideals of beauty have changed overtime, the pursuit of a flawless appearance has always been aimed for by humans. An essential part of a beautiful and attractive appearance is the condition and complexion of the skin.

In order for the skin to be able to perform the full range of its biological functions, it requires regular cleansing and care. Cleansing of the skin serves to remove dirt, sweat and residual dead skin particles, which form an ideal nutrient source for all kinds of germs and parasites. Skincare products mostly serve for moisturizing and refatting the skin. Active ingredients are commonly added thereto, which are intended to regenerate the skin and for example to prevent and reduce the premature aging thereof (for example the appearance of fine lines and wrinkles) or protect against the adverse effects of UV radiation. Skincare products are usually offered in the form of emulsions in which an outer water phase comprises stable finely distributed oil droplets (O/W emulsion) or stable finely distributed water droplets are present in a continuous oil phase (W/O emulsion).

Emulsions for skincare are generally marketed as ready-to-use products. However, these products have the disadvantage that they have a high weight due to their more or less high water content. Especially during transport, the high weight results in a relatively high energy and fuel consumption.

It was therefore the object of the present invention to develop a cosmetic emulsion in which the weight of the preparation is reduced and which, during transport especially of relatively large container quantities, results in a lower energy and/or fuel consumption.

Most cosmetic emulsions are produced by the industrial manufacturer of the products using the “hot-hot” or “hot-cold” process, in which a hot oil phase, generally above 80° C., is blended with a likewise heated (“hot-hot”) or room-temperature (“hot-cold”) water phase with stirring, homogenized and subsequently cooled. All of these production process steps are associated with a relatively high energy consumption. It was therefore the object of the present invention to develop a cosmetic emulsion that has a reduced energy consumption during industrial production.

The achievement of both objects should moreover significantly reduce the “CO₂ footprint”, i.e. the CO₂ emissions associated with the production and transport of the products.

Last but not least, conventional cosmetic finished products are often perceived by consumers as boring to use. The desire (or else the “play instinct”) of many consumers to be involved in the production of cosmetics themselves, and the general consumer desire for products with at most a low “CO₂ footprint”, can be met by conventional finished products only to a limited extent.

It was therefore the object of the present invention to develop a cosmetic product that meets these consumer desires.

These objects are surprisingly achieved by a process for producing a cosmetic water-in-oil emulsion, where a glycerin-in-oil emulsion comprising
a) glycerin,
b) one or more lipids that are liquid at room temperature and standard pressure,
c) one or more waxes,
d) one or more salts,
e) polyglyceryl-3 polyricinoleate (INCI: Polyglyceryl-3 Polyricinoleate),
f) one or more preservatives,
is admixed with water in a storage container and shaken or stirred.

The glycerin-in-oil emulsion according to the invention makes it possible for the user/consumer to produce a stable, ready-to-use W/O emulsion for skin and body care by simple addition of water with brief shaking, without having to use a technically complex or time-intensive production process.

Although there has been no lack of attempts in the past to develop low-water or even water-free “pre-emulsions” and to offer them to the consumer for the production of a care emulsion in the home environment, the production was relatively complex either because it was necessary for example to use warm water or because the use of complex equipment such as agitators (“mixers”) was required, or the final product produced at home left much to be desired in terms of quality because an actually finely dispersed, homogeneous emulsion was not formed. The regionally varying salt content of the water (for example the “water hardness”) also caused difficulties since this has a significant influence on the formation and stability of an emulsion. What worked in Hamburg could not necessarily be expected to succeed in Munich.

The German patent application DE 102022207466.1, which was filed on 7.21.2022 and has been further developed with the present invention, discloses preparations that still have the disadvantage of having a relatively great increase in viscosity in the case of a prolonged storage period. This phenomenon also occurs in preparations in which the preservative is incorporated into the oil phase.

It was therefore the object of the present invention to develop a cosmetic product that has a more or less constant viscosity in the case of a prolonged storage period and is thus more viscosity-stable.

This object is achieved by the addition of preservatives to the glycerin phase. This solution was not obvious because, from a microbial point of view, it is not necessary to preserve the glycerin phase or the glycerin.

Within the scope of the present disclosure, the phrases “according to the invention” etc. always relate to the process according to the invention and to the product of the process.

It is particularly advantageous according to the invention for the processes according to the invention if the preparation comprises at most 0.5% by weight of water, based on the weight of the glycerin-in-oil emulsion. This water is not added directly, but rather is introduced into the preparation in conjunction with raw materials that comprise small amounts of water. A higher content of water can destabilize the glycerin-in-oil emulsion and lead to phase separation. A stable W/O emulsion system is formed again only at relatively high water concentrations (greater than 30% by weight), as occurs in the W/O emulsion produced by the process according to the invention.

It is therefore preferred according to the invention if the glycerin used comprises at most 1% by weight, based on the glycerin, of water. Use is ideally made of 99% or 99.5% pure glycerin.

It is advantageous according to the invention if alkaline earth metal salts are used as salts in the glycerin-in-oil emulsion.

Embodiments of the present invention that are preferred according to the invention are characterized in that magnesium sulfate is used as salt in the glycerin-in-oil emulsion.

Moreover, it is particularly preferred according to the invention if magnesium sulfate heptahydrate (MgSO₄·7H₂O) is used as magnesium sulfate. This may seem surprising at first glance, but anhydrous magnesium sulfate is much more difficult to incorporate into the glycerin-in-oil emulsion than magnesium sulfate containing water of crystallization. Anhydrous magnesium sulfate dissolves less well in glycerin and tends to form lumps.

It is advantageous according to the invention if one or more compounds selected from the group of caprylic/capric acid triglyceride (INCI: Caprylic/Capric Triglyceride), ethylhexyl stearate (INCI: Ethylhexyl Stearate), triisostearin (INCI: Triisostearin), coco-caprylate/caprate (INCI: CocoCaprylate/Caprate), octyldodecanol, cocoglycerides (INCI: Cocoglycerides), vegetable oil (INCI: Vegetable Oil), Dicaprylyl Ether, sunflower oil (INCI: Helianthus Annuus Seed Oil) are used as lipids that are liquid at room temperature and standard pressure.

The use of caprylic/capric acid triglyceride (INCI: Caprylic/Capric Triglyceride), coco-caprylate/caprate (INCI: Coco-Caprylate/Caprate) and vegetable oil (INCI: Vegetable Oil) is preferred according to the invention here.

If the oil phase of the glycerin-in-oil emulsion comprises caprylic/capric acid triglyceride (INCI: Caprylic/Capric Triglyceride), then according to the invention this compound is preferably used in a concentration of 10% to 50% by weight, based on the total weight of the glycerin-in-oil emulsion.

If the oil phase of the glycerin-in-oil emulsion comprises coco-caprylate/caprate (INCI: CocoCaprylate/Caprate), then according to the invention this compound is preferably used in a concentration of 10% to 50% by weight, based on the total weight of the glycerin-in-oil emulsion.

If the oil phase of the glycerin-in-oil emulsion comprises vegetable oil for example based on rapeseed oil (INCI: Vegetable Oil), then according to the invention this compound is preferably used in a concentration of 10% to 50% by weight, based on the total weight of the glycerin-in-oil emulsion.

It is advantageous according to the invention if one or more compounds selected from the group of hydrogenated rapeseed oil (INCI: Hydrogenated Rapeseed Oil), hydrogenated castor oil (INCI: Hydrogenated Castor Oil), hydrogenated vegetable oil (INCI: Hydrogenated Vegetable Oil), glyceryl stearate (INCI: Glyceryl Stearate) are used as wax. The use of hydrogenated rapeseed oil (INCI: Hydrogenated Rapeseed Oil) is preferred according to the invention here.

If the oil phase of the glycerin-in-oil emulsion comprises hydrogenated rapeseed oil (INCI: Hydrogenated Rapeseed Oil), then according to the invention this compound is preferably used in a concentration of 0.5% to 5% by weight, based on the total weight of the glycerin-in-oil emulsion.

The embodiments of the present invention that are advantageous according to the invention are characterized in that the preparation comprises the glycerin in an amount of 20% to 40% by weight, based on the total weight of the glycerin-in-oil emulsion.

The embodiments of the present invention that are advantageous according to the invention are also characterized in that the preparation comprises lipids that are liquid at room temperature and standard pressure of feature b) in a total amount of 30% to 60% by weight, based on the total weight of the glycerin-in-oil emulsion.

Furthermore, it is advantageous according to the invention if the preparation comprises waxes of feature c) in an amount of 0.5% to 5% by weight, based on the total weight of the glycerin-in-oil emulsion.

Moreover, it is advantageous according to the invention if the preparation comprises magnesium sulfate in an amount of 1% to 4% by weight, based on the total weight of the glycerin-in-oil emulsion.

Last but not least, the embodiments of the present invention that are advantageous according to the invention are characterized in that the preparation comprises polyglyceryl-3 polyricinoleate (INCI: Polyglyceryl-3 Polyricinoleate) in an amount of 10% to 20% by weight, based on the total weight of the glycerin-in-oil emulsion.

Moreover, the glycerin-in-oil emulsion according to the invention may comprise further ingredients, such as those usually used in cosmetic emulsions, for example cosmetic active ingredients, UV filters, chelating agents, antioxidants, powder raw materials, etc.

It is advantageous according to the invention if the glycerin-in-oil emulsion according to the invention is contained in a jar or a bottle with a water-impermeable closure that is made of tinplate, glass, polypropylene, polyethylene or polyethylene terephthalate. The filling quantity in this storage container here should, preferably according to the invention, be selected such that this storage container can additionally accommodate three times the amount of water.

It is advantageous according to the invention for the process according to the invention if ajar or a bottle made of tinplate, aluminum, glass, polypropylene, polyethylene or polyethylene terephthalate and having a water-impermeable closure is used as storage container, and the emulsion is formed by shaking.

Embodiments of the process according to the invention that are advantageous according to the invention are also characterized in that the process is performed at a temperature between 10° C. and 30° C.

In addition, it is advantageous according to the invention for the process if the weight ratio of glycerin-in-oil emulsion to water is from 20:80% by weight to 60:40% by weight. It is preferred according to the invention if the weight ratio of glycerin-in-oil emulsion to water is from 30:70% by weight to 50:50% by weight and particularly preferably 1:2.

The use amount of the water makes it possible here to control the viscosity of the W/O emulsion that can be obtained from the glycerin-in-oil emulsion by addition of water. A low content of water (60-75% by weight) results in a high-viscosity preparation, in particular in a cream. By contrast, lower amounts of water (40-60% by weight) form thinner lotions.

Moreover, it is advantageous according to the invention if the water has a temperature of at least 10° C. A water temperature of 15° C. to 25° C. is preferred according to the invention here.

Furthermore, it is one of the embodiments of the process according to the invention that are advantageous according to the invention if the shaking of the storage container is performed by hand and the shaking process has a duration of at most 90 seconds. A duration of the shaking process of 10 to 30 seconds is preferred according to the invention here.

It is advantageous according to the invention if one or more compounds selected from ethylhexylglycerin, hydroxyacetophenone, benzyl alcohol, Glyceryl Caprate, ethanol, Polyglyceryl-2 Caprate, propylene glycol, butylene glycol, 2-methylpropane-1,3-diol, pentane-1,2-diol, hexane-1,2-diol, octane-1,2-diol, glyceryl caprylate, decane-1,2-diol, phenoxyethanol are used as preservative.

It is preferred according to the invention if one or more compounds selected from ethylhexylglycerin, hydroxyacetophenone, benzyl alcohol, Glyceryl Caprate, ethanol, Polyglyceryl-2 Caprate, octane-1,2-diol, decane-1,2-diol are used as preservative.

It is also advantageous in the context of the present invention if the preservative(s) is (are) present in the glycerin-in-oil emulsion in a total amount of 0.7% to 10% by weight, based on the total weight of the glycerin-in-oil emulsion.

The embodiments of the present invention that are advantageous according to the invention here are characterized in that the preservative(s) is (are) incorporated into the glycerin phase of the emulsion.

It is preferred according to the invention here if the preservative(s) is (are) incorporated into the glycerin phase of the glycerin-in-oil emulsion before it is emulsified with the oil phase.

The invention also provides a cosmetic water-in-oil emulsion produced by the process according to the invention.

Advantageously according to the invention, said emulsion is characterized in that it is in the form of a cream or lotion.

### COMPARATIVE EXPERIMENTS

The effect according to the invention was able to be demonstrated by way of example with the following experiment:

The following concentrates (=glycerin-in-oil emulsion) and the activation time until the formation of the water-in-oil emulsion (=product of the process) were determined and the viscosity of these water-in-oil emulsions was investigated over a period of 30 days (at a storage temperature of 25° C.).

Production of the concentrate (=glycerin-in-oil emulsion) in the laboratory: The emulsifiers and lipid-soluble raw materials in the oil phase are melted at 80° C. prior to homogenization until all constituents have reached a liquid state of matter. Constituents of the hydrophilic phase are dissolved in the glycerin using a heatable magnetic stirrer at 75° C. with stirring until the liquid is clear. This glycerin phase is added to the oil phase during the homogenization process (for example with an IKA T25 digital Ultra-Turrax) as soon as both phases have reached a temperature of 75-80° C. The phases are combined at a speed of 8000 rpm. Once the phases have been combined, homogenization is performed at a speed of 11 200 rpm for a further 3 min. The concentrate is then cooled with stirring using the IKA stirrer; a second homogenization is optionally performed after addition of a perfume at approx. 35° C.

Production of the W/O emulsion and determination of the activation time:

To produce the water-containing W/O emulsions and to determine the activation time until formation thereof, first the concentrate (=glycerin-in-oil emulsion) and subsequently the water are weighed into a 500 ml wide-neck bottle in a total amount of 400 g (for example at 30% to 70%: 120 g of concentrate and 280 g of water). The vessel is then securely closed and shaken by hand until the resulting W/O emulsion has a homogeneous appearance and the time is stopped.

If no homogeneous W/O emulsion is formed within 3 minutes of shaking, the batch is terminated.

The viscosity was determined with the following viscometer under the specified conditions. The measurement is performed at 25° C. in a 150 ml rolled rim glass bottle by means of a Rheomat R 123 from proRheo. The Rheomat R 123 from proRheo GmbH is a rotational viscometer, i.e. a measuring body rotates in the substance to be measured. The force required to rotate the measuring body in the sample at a defined speed is measured. The viscosity is calculated from this torque, the speed of the measuring body and the geometric dimensions of the measuring system used. The measuring body used is the No. 1 measuring body (Article No. 200 0191), speed range 62.5 min⁻¹.

| | G 235 | | G 284 | |
|---|---|---|---|---|
| | Concen- | | Concen- | |
| EU INCI | tration | Preparation: | tration | Preparation: |
| Declaration | (%) | Phase division | (%) | Phase division |
| Vegetable Oil | 16.2 | Fat phase | 16.2 | Fat phase |
| Caprylic/Capric Triglyceride | 18 | Fat phase | 18 | Fat phase |
| Coco-Caprylate/Caprate + Tocopherol | 14.5 | Fat phase | 14.5 | Fat phase |
| Hydrogenated Rapeseed Oil | 3 | Fat phase | 3 | Fat phase |
| Polyglyceryl-3 Polyricinoleate | 14 | Fat phase | 14 | Fat phase |
| Glyceryl Caprate | 0.8 | Fat phase | 0.8 | Fat phase |
| Perfume, Limonene, Linalool, Eugenol, | 1 | Perfume | 1 | Perfume phase |
| Citral, Geraniol, Citronellol, | | phase | | |
| Coumarin, Isoeugenol | | | | |
| Glycerin (99%), Aqua (1%) | 30.5 | Glycerin phase | 30.5 | Glycerin phase |
| Caprylyl Glycol (99%), Aqua (1%) | 1 | Fat phase | 1 | Glycerin phase |
| Magnesium Sulfate | 1 | Glycerin phase | 1 | Glycerin phase |
| Activation time of the emulsion | 3-6 seconds | | 3-6 seconds | |
| (35% concentrate + 65% water) | | | | |
| 1 day after preparation | | | | |

| | | |
|---|---|---|
| Vis + 25 [mPas], 1 d | 550 | 400 |
| Vis + 25 [mPas], 7 d | 1000 | 400 |
| Vis + 25 [mPas], 14 d | 1900 | 450 |
| Vis + 25 [mPas], 30 d | 2650 | 500 |

### Formulations and Results

### Examples

### Production of the Concentrate (Glycerin-In-Oil Emulsion)

The amounts can be found in the exemplary formulations in the table.

The fat phase comprising Polyglyceryl-3 Polyricinoleate, the liquid lipids and the wax is weighed into a beaker and heated to 80° in a water bath with repeated stirring using a stirring thermometer. The magnesium sulfate is weighed into a beaker together with the glycerin and dissolved until clear using a magnetic stirrer on a hot plate at 75° C. with vigorous stirring. The preservatives or preservation aids are added to the clear glycerin phase and dissolved or mixed with stirring. The hot glycerin phase is dispersed into the hot fat phase using an Ultra-Turrax at 8000 rpm for 2 minutes. The mixture is then dispersed at 11 200 rpm for a further 3 minutes. This is followed by cooling to 35° C. using an IKA stirrer. The perfume is stirred into the cooled glycerin-in-oil emulsion and the mixture is then homogenized once again for 2 minutes with the Ultra-Turrax at 11 200 rpm.

| | G 205 | G 206 | G 207 | G 208 | G 213 | G 214 | G 215 |
|---|---|---|---|---|---|---|---|
| Perfume 1 | 0.5 | 0.5 | 0.5 | 0.5 | | | |
| Perfume 2 | | | | | 0.5 | 0.5 | 0.5 |
| Glyceryl Caprylate | 0.8 | | | | | | |
| Caprylyl Glycol | | 1 | | | 1 | 1 | 1 |
| (99%), Aqua (1%) | | | | | | | |
| Polyglyceryl-2 | | | 2.8 | | | 1.4 | |
| Caprate | | | | | | | |
| Glyceryl Caprate | | | | 0.8 | 0.8 | | |
| Pentylene Glycol | | | | | | | 2.8 |
| (99%), Aqua (1%) | | | | | | | |
| Vegetable Oil | 16.7 | 16.5 | 16 | 16.7 | 16.2 | 16.1 | 15.0 |
| Caprylic/Capric | 18 | 18 | 17.7 | 18 | 18 | 18 | 17.7 |
| Triglyceride | | | | | | | |
| Coco-Caprylate/ | 16 | 16 | 15.0 | 16 | 15.5 | 15.0 | 15.0 |
| Caprate | | | | | | | |
| Hydrogenated | 3 | 3 | 3 | 3 | 3 | 3 | 3 |
| Rapeseed Oil | | | | | | | |
| Glycerin (99%), | 28.2 | 28.2 | 28.2 | 28.2 | 28.2 | 28.2 | 28.2 |
| Aqua (1%) | | | | | | | |
| Polyglyceryl-3 | 14 | 14 | 14 | 14 | 14 | 14 | 14 |
| Polyricinoleate | | | | | | | |
| Magnesium Sulfate | 2.8 | 2.8 | 2.8 | 2.8 | 2.8 | 2.8 | 2.8 |
| Vis + 25 [mPas], 1 d | 200 | 300 | 300 | 400 | 300 | 1550 | 400 |
| Vis + 25 [mPas], 7 d | 300 | 450 | 550 | 400 | 350 | 1700 | 350 |
| Vis + 25 [mPas], 14 d | 450 | 900 | 1050 | 850 | 350 | 1750 | 350 |
| Vis + 25 [mPas], 28 d | 1800 | 1400 | 1400 | 1300 | 1100 | 2200 | 1250 |
| Vis + 25 [mPas], 60 d | 1750 | 1450 | 1350 | 1450 | 1350 | 2000 | 1900 |

| | G 216 | G 217 | G 218 | G 219 | G 220 | G 222 |
|---|---|---|---|---|---|---|
| Perfume 2 | 0.5 | 0.5 | 0.5 | | | |
| Perfume 3 | | | | 0.5 | 0.5 | 0.175 |
| Caprylyl Glycol | | 1 | | 1 | 1 | |
| (99%), Aqua (1%) | | | | | | |
| Polyglyceryl-2 | | | | | 1.4 | |
| Caprate | | | | | | |
| Glyceryl Caprate | 0.8 | | 0.8 | 0.8 | | 0.8 |
| Pentylene Glycol | 2.8 | | | | | 2.8 |
| (99%), Aqua (1%) | | | | | | |
| Vegetable Oil | 15.0 | 16.5 | 16.7 | 16.2 | 16.1 | 15.0 |
| Caprylic/Capric | 17.9 | 18 | 18 | 18 | 18 | 17.9 |
| Triglyceride | | | | | | |
| Coco-Caprylate/ | 15.0 | 16 | 16 | 15.5 | 15.0 | 15.0 |
| Caprate | | | | | | |
| Hydrogenated | 3 | 3 | 3 | 3 | 3 | 3 |
| Rapeseed Oil | | | | | | |
| Glycerin (99%), | 28.2 | 28.2 | 28.2 | 28.2 | 28.2 | 28.2 |
| Aqua (1%) | | | | | | |
| Polyglyceryl-3 | 14 | 14 | 14 | 14 | 14 | 14 |
| Polyricinoleate | | | | | | |
| Magnesium Sulfate | 2.8 | 2.8 | 2.8 | 2.8 | 2.8 | 2.8 |
| Vis + 25 [mPas], 1 d | 300 | 550 | 950 | 1050 | 1100 | 1200 |
| Vis + 25 [mPas], 7 d | 400 | 700 | 1200 | 1300 | 1350 | 1450 |
| Vis + 25 [mPas], 14 d | 350 | 1050 | 1950 | 2050 | 2100 | 2400 |
| Vis + 25 [mPas], 28 d | 800 | 1300 | 2050 | 2200 | 2250 | 2600 |
| Vis + 25 [mPas], 60 d | 1500 | 1350 | 2150 | 2350 | 2300 | 2600 |

1. A process for producing a cosmetic water-in-oil emulsion, where a glycerin-in-oil emulsion comprising
a. glycerin,
b. one or more lipids that are liquid at room temperature and standard pressure,
c. one or more waxes,
d. one or more salts,
e. polyglyceryl-3 polyricinoleate (INCI: Polyglyceryl-3 Polyricinoleate),
f. one or more preservatives,
is admixed with water in a storage container and shaken or stirred.

2. The process as claimed in claim 1, wherein the glycerin-in-oil emulsion comprises at most 0.5% by weight of water, based on the total weight of the glycerin-in-oil emulsion.

3. The process as claimed in claim 1, wherein alkaline earth metal salts are used as salts in the glycerinin-water emulsion.

4. The process as claimed in claim 1, wherein magnesium sulfate is used as salt in the glycerin-in-oil emulsion.

5. The process as claimed in claim 1, wherein the glycerin used comprises at most 1% by weight, based on the glycerin, of water.

6. The process as claimed in claim 4, wherein magnesium sulfate heptahydrate (MgSO₄·7H₂O) is used as magnesium sulfate.

7. The process as claimed in claim 1, wherein one or more compounds selected from the group of caprylic/capric acid triglyceride (INCI: Caprylic/Capric Triglyceride), ethylhexyl stearate (INCI: Ethylhexyl Stearate), triisostearin (INCI: Triisostearin), coco-caprylate/caprate (INCI: CocoCaprylate/Caprate), octyldodecanol, cocoglycerides (INCI: Cocoglycerides), vegetable oil (INCI: Vegetable Oil), Dicaprylyl Ether, sunflower oil (INCI: Helianthus Annuus Seed Oil) are used as lipids that are liquid at room temperature and standard pressure.

8. The process as claimed in claim 1, wherein one or more compounds selected from the group of hydrogenated rapeseed oil (INCI: Hydrogenated Rapeseed Oil), hydrogenated castor oil (INCI: Hydrogenated Castor Oil), hydrogenated vegetable oil (INCI: Hydrogenated Vegetable Oil), glyceryl stearate (INCI: Glyceryl Stea-rate) are used as wax.

9. The process as claimed in claim 1, wherein the preparation comprises the glycerin in an amount of 20% to 40% by weight, based on the total weight of the glycerin-in-oil emulsion.

10. The process as claimed in claim 1, wherein the preparation comprises lipids that are liquid at room temperature and standard pressure of feature b) in a total amount of 30% to 60% by weight, based on the total weight of the glycerin-in-oil emulsion.

11. The process as claimed in claim 1, wherein the preparation comprises waxes of feature c) in an amount of 0.5% to 5% by weight, based on the total weight of the glycerin-in-oil emulsion.

12. The process as claimed in claim 1, wherein the preparation comprises magnesium sulfate in an amount of 1% to 4% by weight, based on the total weight of the glycerin-in-oil emulsion.

13. The process as claimed in claim 1, wherein the preparation comprises polyglyceryl-3 polyricinoleate (INCI: Polyglyceryl-3 Polyricinoleate) in an amount of 10% to 20% by weight, based on the total weight of the glycerin-in-oil emulsion.

14. The process as claimed in claim 1, wherein the storage container is a jar or a bottle made of tinplate, aluminum, glass, polypropylene, polyethylene or polyethylene terephthalate, having a water-impermeable closure, and
the glycerin-in-oil emulsion admixed with the water in the storage container is shaken.

15. The process as claimed in claim 1, wherein the process is performed at a temperature between 10° C. and 30° C.

16. The process as claimed in claim 1, wherein the weight ratio of glycerin-in-oil emulsion to water is from 20:80% by weight to 60:40% by weight.

17. The process as claimed in claim 1, wherein the water has a temperature of at least 10° C.

18. The process as claimed in claim 1, wherein the water has a temperature of 15° C. to 25° C.

19. The process as claimed in claim 1, wherein the shaking of the storage container is performed by hand and the shaking process has a duration of at most 90 seconds.

20. The process as claimed in claim 1, wherein one or more compounds selected from ethylhexylglycerin, hydroxyacetophenone, benzyl alcohol, Glyceryl Caprate, ethanol, Polyglyceryl-2 Caprate, propylene glycol, butylene glycol, 2-methylpropane-1,3-diol, pentane-1,2-diol, hexane-1,2-diol, octane-1,2-diol, glyceryl caprylate, decane-1,2-diol, phenoxyethanol are used as preservative.

21. The process as claimed in claim 1, wherein one or more compounds selected from ethylhexylglycerin, hydroxyacetophenone, benzyl alcohol, Glyceryl Caprate, ethanol, Polyglyceryl-2 Caprate, octane-1,2-diol, decane-1,2-diol are used as preservative.

22. The process as claimed in claim 1, wherein the preservative(s) is (are) present in the glycerin-in-oil emulsion in a total amount of 0.7% to 10% by weight, based on the total weight of the glycerinin-oil emulsion.

23. The process as claimed in claim 1, wherein the preservative(s) is (are) incorporated into the glycerin phase of the emulsion.

24. The process as claimed in claim 1, wherein the preservative(s) is (are) incorporated into the glycerin phase of the glycerin-in-oil emulsion before it is emulsified with the oil phase.

25. A cosmetic water-in-oil emulsion produced by a process as claimed in claim 1.

26. The water-in-oil emulsion as claimed in claim 25, wherein it is in the form of a cream or lotion.
